(19) United States
(12) Patent Application Publication
(10) Pub. No.: US 2006/0019872 A1
(43) Pub. Date: Jan. 26, 2006

(54) Oral formulations for poorly absorptive hydrophilic drugs

(75) Inventors: Chung-Il Hong, Chicago, IL (US); Hee-Jong Shin, Wonmi-gu (KR); Min-Hyo Ki, Cheonan-si (KR); Mee-Hwa Choi, Cheonan-si (KR)

Correspondence Address: VENABLE LLP, P.O. BOX 34385, WASHINGTON, DC, 20045-9998, US

(73) Assignee: Chong Kun Dang Pharmaceutical Corp., Seoul (KR)

(21) Appl. No.: 10/538,686

(22) PCT Filed: Dec. 10, 2003
(86) PCT No.: PCT/KR03/02700

§ 371 Date: Jun. 10, 2005

(30) Foreign Application Priority Data
Dec. 11, 2002 (KR) 10-2002-0078778

(51) Int. Cl. A61K 38/28; A61K 38/14; A61K 31/727; A61K 31/545

(52) U.S. Cl. 514/3; 514/12; 514/8; 514/56; 514/200

(57) ABSTRACT

Disclosed herein is a pharmaceutical composition for oral absorption of polar drugs. The composition consists essentially of (a) at least one polar active substance having a bioavailability of less than 30% which is poorly absorptive through lipid membranes because of its high hydrophilicity and charged ion; (b) at least one organic alkalizing agent having an amino acid or polyol structure which shows alkalinity in aqueous solution and is ionically bonded to the polar active substance; and (c) at least one surfactant having a C₆₋₁₈ fatty acid structure which has an HLB of 4 to 18. Alternatively, the composition consists essentially of (d) at least one organic alkalizing agent having the characteristics of both the organic alkalizing agent and the surfactant instead of the organic alkalizing agent and the surfactant. The composition enables polar active drugs to penetrate the gastro-intestinal membrane and oral dosage forms to be substituted for injections.

### TECHNICAL FIELD

The present invention relates to a pharmaceutical composition suitable for oral absorption of hydrophilic drugs, and more particularly to a novel pharmaceutical composition suitable for oral absorption of charged and highly polar active substances which are nearly impossible to penetrate lipid membranes.

### BACKGROUND ART

Among drugs developed hitherto around the world, some drugs are hardly absorbed via the oral route due to their poor solubility (non-polarity), whereas some drugs hardly penetrate the lipid membranes and are thus orally unabsorbed due to their high polarity. Examples of highly polar or highly ionizable drugs in aqueous solutions include injectable antibiotics and anticancer agents, peptide-based and protein-based drugs. Most of these drugs have not been formulated for oral absorption yet.

Since highly polar drugs are seldom subjected to free diffusion and penetration, which are main absorption mechanisms through the lipid membranes of the gastro-intestinal tract, it has been recognized that the polar drugs are administered almost exclusively by intravenous, intramuscular and subcutaneous injections. Some drugs can be absorbed by a transporter, such as a dipeptide transporter, specific to the biological membranes, although they are too highly polar to penetrate the lipid membranes. However, most of the highly polar drugs are very limited in their ability to pass the lipid membranes. Thus, the present inventors have earnestly and intensively conducted research with the aim of developing oral formulations of polar drugs. As a result, the present inventors designed a pharmaceutical composition which allows spontaneous formation of polarity-reduced and hydrophobic particles, and passive diffusion and distribution of the hydrophobic particles in gastro-intestinal tract.

Recent studies on improving the oral absorption of hardly soluble drugs have focused on the increase in the solubility of the drugs in order to increase the oral absorption rate. In this connection, the use of a surfactant for increasing the solubility of poorly water-soluble or insoluble active substances is described in patent literatures, including German Patent No. 4,003,844 and U.S. Pat. No. 3,882,243. However, these prior arts are distinguished from the present invention in that the active substance used in the present invention is polar and relatively hydrophilic material and has a partition coefficient (Log P) of 1.5 or less, and preferably 1.0 or less.

On the other hand, U.S. Patent Laid-open No. 2002-015730, European Patent No. 230,332, Korean Patent Laid-open No. 2001-0042083, Korean Patent No. 103209, PCT Publication WO 00/25,598, etc. disclose formulation designs for changing the release characteristics of drugs having excellent oral absorption in itself. According to these prior arts, an oil or surfactant containing a fatty acid moiety in its structure is added to these drugs for controlled release from dosage forms. That is, the object of these prior arts resides in the control of the release rate of active substances from the formulations, regardless of increase in the oral absorption rate. Accordingly, the present invention is clearly different from the prior arts in terms of its object.

Further, techniques concerning increase in the oral absorption rate by adding a surfactant with an organic acid- or fatty acid structure to a polar drug, such as a peptide-based drug, are described in U.S. Pat. Nos. 5,929,027, 5,665,711, 5,318,781 and 4,397,951, PCT publication WO 94/25062, Korean Patent No. 026,778 and so forth. Techniques for facilitating rectal, vaginal or nasal administration by addition of a surfactant such as a sugar ester to a polar drug, are described in European Patent Nos. 983,769 and 702,958, Korean Patent Laid-open No. 2001-0006361, Korean Patent No. 020,298 and the like. These prior art techniques are similar to the composition of the present invention in that a surfactant having a fatty acid structure is used to increase the penetration rate of polar drugs, such as peptides, through the lipid membranes. However, the surfactant having a fatty acid structure is added simply to increase the absorption of drugs. In contrast to these prior arts, the formation of a relatively hydrophobic conjugate (particle) containing a polar drug is described as a critical technique of the present invention. The prior arts fail to mention the critical technique. Specifically, the present invention is highly distinguished from the prior arts in that an organic alkalizing agent is combined to a polar drug to form a hydrophobic conjugate in which charges are neutralized, thereby providing conditions advantageous for the penetration of the drug through the lipid membranes. The biological membranes of the gastrointestinal tract have a phospholipid bilayer structure. Accordingly, so long as an enzyme present in the biological membranes does not specifically transport a polar drug, the polar drug does not easily pass through the lipid membranes due to charges of the polar drug. Accordingly, there is a need to neutralize charges disadvantageous for absorption through the lipid membranes, reduce the polarity and relatively increase the hydrophobicity of polar drugs, thus inducing passive diffusion of the polar drugs through the lipid membranes.

### DISCLOSURE OF THE INVENTION

Since conventional highly polar active substances cannot penetrate the lipid membranes of the gastrointestinal tract, they are administered almost exclusively by injection. Therefore, the present invention has been made in view of the above problems, and it is an object of the present invention to provide a novel pharmaceutical composition suitable for oral absorption of highly polar active substances. Specifically, the pharmaceutical composition comprises a polar active substance of which penetration through lipid membranes is nearly impossible, an organic alkalizing agent for neutralizing the charge of the polar active substance and reducing the polarity of the polar active substance, and a surfactant having a fatty acid structure. If necessary, instead of the organic alkalizing agent, and the surfactant, another alkalizing agent having the characteristics of both the organic alkalizing agent and the surfactant may be used to increase the oral absorption rate.

In order to accomplish the above object of the present invention, there is provided a pharmaceutical composition for oral absorption of a polar active substance, consisting essentially of:
(a) at least one polar active substance having a bioavailability of less than 30% which is poorly absorptive through lipid membranes because of its high hydrophilicity and charged ion.
(b) at least one organic alkalizing agent having an amino acid or polyol structure which shows alkalinity in aqueous solution and is ionically bonded to the polar active substance; and
(c) at least one surfactant having a C₆₋₁₈ fatty acid structure which has an HLB (Hydrophilic-Lipophilic Balance) value of 4 to 18.

If necessary, instead of the organic alkalizing agent and the surfactant, (d) at least one organic alkalizing agent having the characteristics of both the organic alkalizing agent and the surfactant may be used. The alkalizing agent of (d) shows alkalinity in aqueous solution and is ionically bonded to the polar active substance. The alkalizing agent of (d) is selected from those having a fatty acid ester structure.

According to the present invention, the anionic moiety of the polar active substance (drug) is ionically bonded to the cationic moiety of the organic alkalizing agent to neutralize the charge, thereby forming a relatively hydrophobic conjugate. The hydrophobic conjugate thus formed is bound with the surfactant having a fatty acid structure, and thus enables the transport of the drug through the lipid membranes.

Specifically, according to the present invention, the polarity of active substances is reduced, the charge of active substances is neutralized, and the free diffusion and the distribution of active substances are induced, thereby accomplishing a remarkable increase in the non-specific absorption of the active substances through the lipid membranes. Hence, even active substances having a partition coefficient (Log P) of about 1.5 or less and preferably about 1 or less for which passage through the lipid membranes is nearly impossible due to their high polarity, can be orally absorbed. The partition coefficient (Log P) is calculated in accordance with the following method. First, a drug is dissolved in a mixed solution (1:1) of octanol and water. When phase separation takes place, concentrations of the drug dissolved in each phase are measured. Logarithms are taken on the relative value of the measured concentrations to calculate a partition coefficient (Log P) of the drug, which is given by Equation 1 below:
Log P=Log(C_{octanol}/C_{water}) Equation 1
wherein C_{octanol} represents the concentration of the drug dissolved in the octanol layer, and
C_{water} represents the concentration of the drug dissolved in the water layer.

The higher the Log P value is, the higher the hydrophobicity (lipophilicity) of a drug is. The lower the Log P value is, the higher the hydrophilicity of a drug is. As is well known in the art, all substances can be empirically expressed by Log P values.

The present invention can be explained in terms of the following two critical techniques. The first critical technique is characterized in that the anionic moiety of the active substance (drug) is ionically bonded to the cationic moiety of the organic alkalizing agent. Accordingly, the charge of the active substance is neutralized to form relatively hydrophobic units composed of the active substance and the organic alkalizing agent. These hydrophobic units are agglomerated with each other to form a relatively hydrophobic conjugate, which is a thermodynamically stabilized form in an aqueous phase (outer phase). The hydrophobic units and the hydrophobic conjugate of the hydrophobic units are schematically shown in FIG. 1. As shown in FIG. 1, since the hydrophobic conjugate has relatively reduced water-solubility and polarity, supplies conditions advantageous for free diffusion and distribution through the lipid membranes are provided.

The second critical technique is characterized in that the surfactant having a fatty acid structure is added to the hydrophobic conjugate to transport the drug through the lipid membranes. Persons skilled in the art can easily anticipate that the nano-sized hydrophobic conjugate enables the oral absorption of the drug. In fact, the hydrophobic conjugate never contributes to oral absorption of the drug. In order to solve this problem, the surfactant having a fatty acid structure is added to the hydrophobic conjugate to transport the drug through the lipid membranes. As shown in FIG. 1, since the surfactant consists of a hydrophobic fatty acid moiety and a non-ionic hydrophilic moiety, it increases the surface activity between the conjugate and the lipid membranes without negatively affecting the ionic bonds formed in the conjugate, and induces the oral absorption of the drug through the lipid membranes. In addition, since the surfactant makes the hydrophobic conjugate small and stable, it provides conditions advantageous for the penetration of the drug through the biological membranes.

In conclusion, the combination of the two critical techniques enables a great increase in the oral absorption rate of polar active substances which have been impossible to administer via the oral route, which creates high value-added technologies. At the same time, the present invention removes inconvenience in connection with the use of injections and thus ensures convenient use for patients.

The composition of the present invention essentially comprises the conjugate composed of the polar active substance and the functional materials with the organic alkalizing moiety, and the surfactant moiety having a fatty acid structure. If necessary, the composition of the present invention may further comprise at least one pharmaceutically acceptable excipient.

The polar active substance is poorly absorbed due to its high hydrophilicity, high water-solubility compared to lipid-solubility. The term “polar active substance” used herein refers to a drug having a bioavailability of less than 30%, and preferably less than 10%. When the polar active substance is dissolved in water it contains one or more anions. In addition, the polar active substance has a partition coefficient (Log P) of 1.5 or lower, and has higher affinity for water than for oil. The polar active substance includes water-soluble antibiotics, anticancer agents, peptide-based drugs, protein-based drugs and polysaccharide-based drugs. Typical examples of the polar active substance are cephaloridine, ceftiofur, cefixime, cefepime, cefoperazone, cefotaxime, ceftazidime, ceftriaxone, moxalactam, gentamicin, aztreonam, amikacin, isepamycin, netilmicin, tobramycin, vancomycin, daptomycin, teicoplanin, polymixin-B, bacitracin, heparin, parathyroid hormone (PTH), growth hormone, insulin and the like. On the other hand, it is known that active substances such as ampicillin, amoxicillin, cephalexin and cefaclor are water-soluble and contain one or more anions upon dissolved in water, but are specifically absorbed by the peptide transporter (PepT1 and PepT2) located on the lipid membranes of the gastro-intestinal tract. So active substance in the present invention is limited to only injectable drugs due to the relatively high hydrophilicity, not orally well-absorptive drugs by means of specific transport though polar and excessively hydrophilic.

Unlike inorganic alkalizing agents, since the organic alkalizing agent used in the present invention contains at least one positive ion (cation), it can ionically bond to the active substance. Concomitantly, the organic alkalizing agent has a partial hydrophobic moiety or non-ionic hydrophilic moiety within its molecular structure. Accordingly, the charge interaction between active substance and the organic alkalizing agent in the present invention enables the shielding of the charge of active substances and the formation of neutralized and relatively hydrophobic conjugate. As used herein, the term “organic alkalizing agent” refers to an organic substance, which shows alkalinity upon dissolved in water and has a relative hydrophobic moiety in its structure. The organic alkalizing agent can have an amino acid, polyol or fatty acid ester structure.

Representative examples of the organic alkalizing agent having an amino acid structure are basic amino acids, such as arginine, lysine and histidine, and derivatives thereof. These basic amino acids may be used alone or in combination as the organic alkalizing agent. An alkanol is bonded with a carboxylic group at the alpha-position of the amino acids to be subjected to dehydration, thereby forming amino acid alkyl esters. Since these amino acid alkyl esters have at least one amine group, they can be used as organic alkalizing agents having amino acid structure. The alkyl group of the amino acid alkyl esters preferably has 12 or fewer carbon atoms, and more preferably 6 or fewer carbon atoms. Specific examples of the amino acid alkyl esters include glycine alkyl esters, alanine alkyl esters, leucine alkyl esters, tyrosine alkyl esters, phenylalanine alkyl esters, tryptophan alkyl esters, arginine alkyl esters, lysine alkyl esters, histidine alkyl esters and the like. Also, at least one substance selected among peptides in which two or more amino acids are joined by a peptide bond, which can show alkalinity in aqueous solution due to the presence of a basic amino acid, may be used as the organic alkalizing agent.

The organic alkalizing agent having a polyol structure having at least one hydroxyl group includes alkaline saccharides, e.g., glucosamine, mannosamine and galactosamine, and oligomers and polymers prepared from 20 or fewer alkaline saccharides as monomers. The organic alkalizing agent includes monoetheanolamine, triethanolamine, diisopropanolamine and choline, all of which have an alkanolamine structure. In addition, saccharide-like meglumine is within the scope of the organic alkalizing agent. These substances may be used alone or in combination as the organic alkalizing agent.

The organic alkalizing agent having a fatty acid ester structure refers to an alkaline substance obtainable from the dehydration between a carboxyl group (—COOH) of an amphoteric compound and a hydroxyl group (—OH) of a fatty acid ester. The term “amphoteric compound” used herein represents a compound having both an amine group (—NH₂) and a carboxyl group (—COOH), which shows both acidity and alkalinity upon dissolved in water. Suitable examples of the amphoteric compound include amino acids and amino fatty acids. The fatty acid ester includes fatty acid esters in which fatty acids having 24 or fewer carbon atoms and preferably 12 or fewer carbon atoms are bonded with glycerol, propylene glycol, or other polyhydric alcohols by the esterification. Also the fatty acid herein has one or more hydroxyl group, e.g., mono-, di-glycerol fatty acid ester, and propylene glycol fatty acid ester. The carboxylic group of the amphoteric compound and the hydroxyl group of the fatty acid ester are subjected to dehydration to form an ester bond. At this time, since the amphoteric compound has at least one charged amine group, it shows alkalinity in an aqueous solution. The organic alkalizing agent having a fatty acid ester structure includes 1-decanoyl-3-lysine glycerol (decanoic acid 3-(2,6-diamino-hexanoyloxy)-2-hydroxy-propyl ester), 1-dodecanoyl-3-arginine glycerol (dodecanoic acid 3-(2-amino-5-guanidinopentanoyloxy)-2-hydroxy-propyl ester), 1-decanoyl-2-lysine propylene glycol (decanoic acid 1-(2,6-diamino-hexanoyloxymethyl)-propylester), 1-dodecanoyl-2-arginine propylene glycol (dodecanoic acid 1-(2-amino-5-guanidinopentanoyloxymethyl)-propylester), etc. These compounds may be used alone or in combination as the organic alkalizing agent. In particular, the organic alkalizing agent having a fatty acid ester structure has surface activity due to its structural characteristics. Accordingly, the use of the organic alkalizing agent having a fatty acid ester structure in the pharmaceutical composition of the present invention eliminates the need of the surfactant having a fatty acid structure.

It is preferred that the surfactant has a C₆₋₁₈ fatty acid structure and has an HLB (Hydrophilic-Lipophilic Balance) value ranging from 4 to 18. The hydrophobic moiety of the surfactant consists of a fatty acid chain, and the hydrophilic moiety consists of a polyol portion having at least one hydroxyl group. The polyol portion is selected from saccharides, e.g., sugar and saccharin; polyhydric alcohols, e.g., glycerol, propylene glycol and polyethylene glycol; and sorbitans, e.g., sorbitan and polysorbitan. Since the hydrophilic moiety of the surfactant does not contain any charge, it stabilizes the hydrophobic conjugate in aqueous solution without negatively affecting the ionic bonds formed in the conjugate. In addition, the hydrophilic moiety of the surfactant interacts with both the lipid membranes and the conjugate to lower the surface tension and to assist in transport of the active substance through the lipid membranes without any irreversible modification to the biological membranes. Suitable examples of the surfactant used in the present invention are sugar fatty acid esters, saccharin fatty acid esters, glycerol fatty acid esters, propylene glycol fatty acid esters, polyethylene glycol fatty acid esters, sorbitan fatty acid esters, polysorbitan fatty acid esters and the like. These substances may be used alone or in combination as the surfactant.

The composition of the present invention is prepared in accordance with the following procedure. First, an active substance is dissolved in an aqueous solution, and then an organic alkalizing agent is added thereto to form a relatively hydrophobic conjugate. At this time, the charge ratio of the active substance to the organic alkalizing agent is in the range of 10:1˜1:10, and preferably 2:1˜1:2. That is, when the active substance contains one anion and the organic alkalizing agent contains one cation, their charge ratio is identical to their molar ratio. Meanwhile, when the active substance has one anion and the organic alkalizing agent has one or more cations, their charge ratio is identical to or smaller than their molar ratio. The hydrophobic conjugate thus formed is in the form of a particle having a size of 10 nm˜100 μm. In the case where the organic alkalizing agent is a relatively highly hydrophobic agent, e.g., a tryptophan alkyl ester, larger particles are formed. If the larger particles are stored for a long time, they are agglomerated with each other. Accordingly, the choice of an appropriate organic alkalizing agent depending on the kind of the active substance is important. And the conjugate formed in aqueous solution has preferably a size of 10 nm˜10 μm. Thereafter, a surfactant having a fatty acid structure is added to the conjugate to prepare the final composition of the present invention. Since the surfactant forms an emulsion or micelles containing the conjugate, it makes the conjugate small and stable, and at the same time, interacts with both the lipid membranes and the conjugate to induce the transport of the active substance. The weight ratio of the surfactant to the drug ranges from 0.1˜20, and preferably 0.5˜10. However, the use of an alkalizing agent having a fatty acid ester structure as the organic alkalizing agent in the composition of the present invention eliminates the composition of additional surfactant. In addition, the composition of the present invention may further comprise at least one pharmaceutically acceptable excipient commonly used in the art. Suitable examples of the excipients include disintegrating agents, suspending agents, thickening agents, lubricating agents, sweetening agents, plasticizers and preservatives.

The composition of the present invention can be administered in a liquid state such as syrup. If necessary, the composition of the present invention may be suspended or emulsified following lyophilization for administration. Further, the active substance, the organic alkalizing agent, and the surfactant having a fatty acid structure in the solid states may be mixed in the ratios described above in the absence of water. In this case, the composition of the present invention may be formulated into solid dosage forms such as powdery granules, tablets and capsules. And a hydrophobic conjugate containing the active substance is formed by the intestinal juices secreted in the body, at the same time, the active substance is transported through the biological membranes. If the active substance is not protected against the gastric acid, it can be covered with an enteric coating to be formed into enteric coated formulations selected from dry syrups, powdery granules, tablets and capsules. As the enteric coating, at least one enteric coating polymer selected from those commonly used in the art, e.g., hydroxypropylmethyl cellulose acetyl citric acid salts, hydroxypropylmethyl cellulose phthalic acid salts, sodium alginate, Eudragit (the trade name of methacrylic acid) for enteric coating and the like, can be used.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a conceptual diagram showing hydrophobic units composed of an active substance and an organic alkalizing agent used in the present invention, and a hydrophobic conjugate of the hydrophobic units;

FIG. 2 is a graph showing the bioavailability (%) of a composition according to the present invention after administration to the duodenum of test animals, in accordance with Experimental Examples 2 to 7 of the present invention;

FIG. 3 is a graph showing the bioavailability (%) of a composition according to the present invention after administration to the duodenum of test animals, in accordance with Experimental Examples 8 to 11 of the present invention;

FIG. 4 is, a graph showing the bioavailability (%) of a composition according to the present invention after administration to the duodenum of test animals, in accordance with Experimental Examples 12 to 14 of the present invention;

FIG. 5 is a graph showing the bioavailability (%) of a composition according to the present invention after administration to the duodenum of test animals, in accordance with Experimental Examples 15 to 18 of the present invention;

FIG. 6 is a graph showing the bioavailability (%) of a composition according to the present invention after administration to the duodenum of test animals, in accordance with Experimental Examples 19 and 20 of the present invention; and

FIG. 7 is a graph showing the bioavailability (%) of comparative compositions after administration to the duodenum of test animals, in accordance with Comparative Experimental Examples 1 to 4.

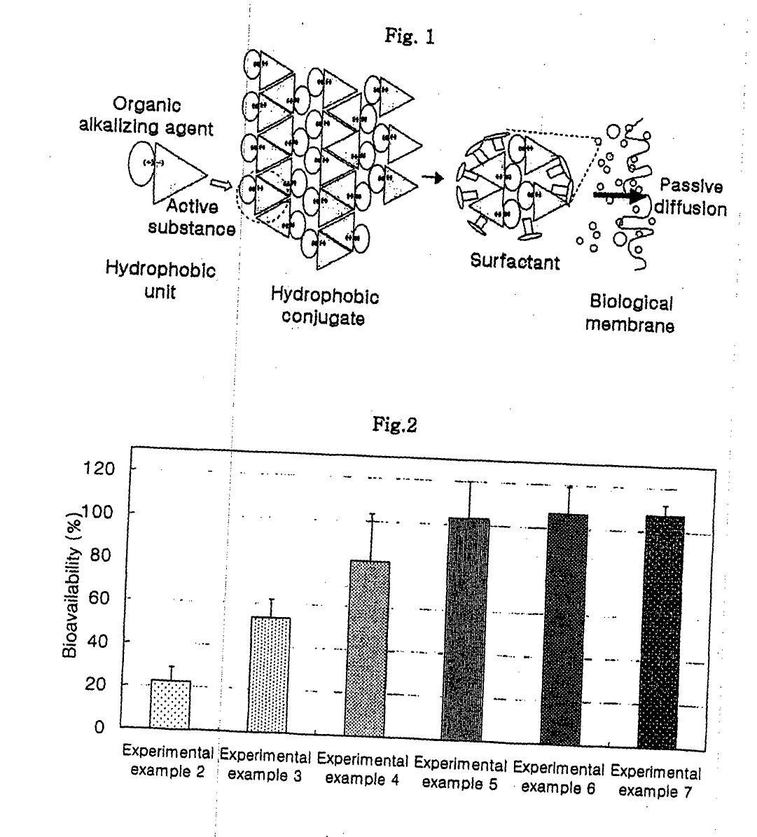

### BEST MODE FOR CARRYING OUT THE INVENTION

The present invention will now be described in more detail with reference to the following examples. However, these examples are given for the purpose of illustration and are not to be construed as limiting the scope of the invention.

### PREPARATION EXAMPLES 1 TO 10

In Preparation Example 1, 1 g of ceftazidime as an active substance (drug) and 273.6 mg of arginine as an organic alkalizing agent were added to 100 ml of water. The resulting mixture was continuously stirred until it became a visually transparent solution.

In Preparation Examples 2 to 6, 1 g of ceftazidime sodium as an active substance, and 215.9 mg of glycine ethyl ester hydrochloride, 307.4 mg of leucine ethyl ester hydrochloride, 360.8 mg of phenylalanine ethyl ester hydrochloride, 422.1 mg of tryptophan ethyl ester hydrochloride and 216.1 mg of arginine ethyl ester hydrochloride, respectively, were added to 100 ml of water, together with different organic alkalizing agents. The resulting mixtures were continuously stirred until they became visually transparent solutions.

In Preparation Example 7, 1 g of ceftazidime as an active substance and 306.7 mg of meglumine as an organic alkalizing agent were added to 100 ml of water. The resulting mixture was continuously stirred until it became a visually transparent solution.

In Preparation Examples 8 to 10, 338.7 mg of glucosamine, 800.0 mg of a chitosan oligomer and 800.0 mg of chitosan, respectively, were added to the solution prepared in Preparation Example 7. The resulting mixtures were continuously stirred until they became visually transparent solutions.

After all transparent solutions prepared above were frozen at −70° C., they were dried in vacuum to prepare dried samples of Preparation Examples 1 to 10, respectively.

### PREPARATION EXAMPLES 11 AND 12

In Preparation Examples 11 and 12, 1 g of ceftazidime sodium as an active substance, and 351.1 mg of 1-decanoyl-3-lysine glycerol.2HCl (decanoic acid 3-(2,6-diamino-hexanoyloxy)-2-hydroxy-propyl ester.2HCl) and 395.1 mg of 1-dodecanoyl-3-arginine glycerol.2HCl (dodecanoic acid 3-(2-amino-5-guanidinopentanoyloxy)-2-hydroxy-propyl ester.2HCl) as organic alkalizing agents, respectively, were added to 100 ml of water. The resulting mixtures were continuously stirred until they became visually transparent solutions. After the transparent solutions were frozen at −70° C., they were dried, in vacuum to prepare dried samples of Preparation Examples 11 and 12, respectively.

### COMPARATIVE PREPARATION EXAMPLES 1 AND 2

In Comparative Preparation Example 1, 120 mg of ceftazidime was added to 5 ml of water, and then 0.2 g of hydroxypropylmethyl cellulose as a suspending agent was added thereto. The resulting mixture was stirred to obtain a suspension of ceftazidime. In Comparative Preparation Example 2, 120 mg of ceftazidime sodium was added to 5 ml of water, and then stirred to obtain a solution of ceftazidime sodium.

### EXAMPLES 1 TO 6, AND COMPARATIVE EXAMPLE 1

In Examples 1 to 3, the dried samples (120 mg as active substances) prepared in Preparation Examples 1, 4 and 6, respectively, were dissolved in 5 ml of water with stirring to obtain solutions.

In Examples 4 to 6, the dried samples (120 mg as active substances) prepared in Preparation Examples 1, 4 and 6, respectively, were dissolved in 5 ml of water with stirring, and then 0.5 g of sugar monolaurate (HLB 16) was dissolved thereto. The sugar monolaurate was employed as a surfactant having a fatty acid structure. The resulting mixtures were dissolved with stirring to obtain solutions.

In Comparative Example 1, the solution obtained in Comparative Preparation Example 2 was used.

The particle size (nm) and the zeta potential (mV) in all solutions obtained above were determined by a particle size analyzer (Zeta PALS, Brookhaven Instrument Corp.). The results are shown in Table 1 below.

TABLE 1

| | Effective diameter (nm) | Zeta potential (mV) |
|---|---|---|
| Example 1 | 597.2 | 1.48 |
| Example 2 | 937.6 | −1.17 |
| Example 3 | 559.8 | −0.03 |
| Example 4 | 16.4 | 0.24 |
| Example 5 | 19.6 | −0.10 |
| Example 6 | 16.2 | −0.13 |
| Comparative Example 1 | Not observed | −1.82 |

As can be seen from Table 1, nano-sized particles having an effective diameter of 100 nm to 1 μm were observed in the solutions of Examples 1 to 3 containing no surfactant, and nano-sized particles having an effective diameter of less than 100 nm were observed in the solutions of Examples 4 to 6 containing surfactants. In addition, the zeta potential values determined in the solutions of Examples 1 to 6 were close to neutral, with slight variations. These results indicate that the cations of the organic alkalizing agents and the anions of the active substances were neutralized. In contrast, although neutralization was made in the transparent solution of Comparative Example 1, but no nano-sized particles were observed therein. In conclusion, it was confirmed that the polar active substances, of which oral absorption is nearly impossible, and the organic alkalizing agents were bonded with each other to form neutralized hydrophobic conjugates in an aqueous solution.

### EXAMPLE 7

1.36 g of the dried sample prepared in Preparation Example 4 and 1.5 g of sugar monopalmitate (HLB 15) as a surfactant were mixed, and then 5% by weight of starch sodium glycolate as a disintegrating agent and 3% by weight of hydroxypropyl cellulose as a binder, based on the total weight of the mixture were added thereto. The disintegrating agent and the binder are pharmaceutically acceptable excipients commonly used in the art. The resulting homogeneous mixture was wet-granulated with water and dried. The dried mixture was sieved using a 20-mesh standard sieve to form granules. After 1% of magnesium stearate as a lubricating agent was added to the granules, the resulting mixture was homogeneously mixed to form final granules.

The final granules were tableted using a tableting machine so that the tablets contained 300 mg of the active substance. Thereafter, a suspension containing 110 mg of a hydroxypropylmethyl cellulose acetyl citric acid salt, 20 mg of triethyl citric acid and 30 mg of talc per one gram of water was used as an enteric coating solution. The enteric coating solution was coated on the tablets using a Hi-coater and dried, so that the tablets had 60 mg of enteric coating formed thereon, based on the dry weight of the tablets.

### EXAMPLE 8

1.22 g of the dried sample prepared in Preparation Example 6 and 1.5 g of sugar monolaurate (HLB 16) as a surfactant were mixed, and then 3% by weight of croscarmellose sodium as a disintegrating agent and 3% by weight of hydroxypropyl cellulose as a binder, based on the total weight of the mixture were added thereto. The disintegrating agent and the binder are pharmaceutically acceptable excipients commonly used in the art. The resulting homogeneous mixture was wet-granulated with water and dried. The dried mixture was sieved using a 20-mesh standard sieve to form granules. After 1% of magnesium stearate as a lubricating agent was added to the granules, the resulting mixture was homogeneously mixed to form final granules.

The final granules were filled into capsules using a capsule-filling machine so that the capsules contained 300 mg of the active substance.

### EXPERIMENTAL EXAMPLE 1

The solution prepared in Comparative Preparation Example 2 was injected into the left jugular vein of a test animal (Sprague-Dawley rat, 6˜8 week old male) in an amount corresponding to 40 mg/kg of the active substance, based on the body weight (kg) of the test animal. 5, 10, 15, 30, 60, 90, 120, 180 and 240 minutes after injection, blood samples (0.6 ml, respectively) were collected from the right jugular vein. The blood plasma was assayed by high performance liquid chromatography. A curve was plotted on the concentrations of the active substance in the blood samples, and the AUC (Area Under Curve, μg-hr/ml) value was calculated using a pharmacokinetics software package (WinNonlin 3.0).

As a result, the AUC value was shown to be 138.90±27.63 (μg-hr/ml), which was used in the calculation of bioavailability.

### EXPERIMENTAL EXAMPLES 2 TO 7

The dried samples prepared in Preparation Examples 1 to 6 were taken in an amount corresponding to 40 mg/kg of the active substances, based on the body weight (kg) of test animals (Sprague-Dawley rats, 6˜8 week old male), and then were dissolved in water with stirring to prepare drug solutions in such amounts that the total dose reached 0.5 ml. The abdomen of the test animals was surgically cut open and a polyethylene tube was inserted into duodenum through the lower portion of the stomach. After 0.5 ml of each drug solution and 0.2 ml of glycerol caprylate were administered into the upper portion of the duodenum through the tube, the abdomen was sutured. 15, 30, 60, 90, 120, 180 and 240 minutes after administration, vein blood samples (0.6 ml, respectively) were collected from the right jugular vein. The blood plasma was assayed by high performance liquid chromatography. A curve was plotted on the concentrations of the active substances in the blood samples, and the AUC (Area Under Curve, μg-hr/ml) value was calculated using a pharmacokinetics, software (WinNonlin 3.0). The bioavailability of the composition was calculated by: Equation 2 below:

$$Bioavailability ( \% ) = \frac{AUC when administered to gastro \text{-} intestinal tract}{AUC when administered intravenously} \times 100 \qquad Equation 2$$

A graph showing the bioavailability (%) of the composition of the present invention after administration to the duodenum of the test animals in accordance with Experimental Examples 2 to 7, is shown in FIG. 2. As is apparent from the graph, the composition of the present invention exhibited a high bioavailability ranging from 20 to 110%. That is, the composition of the present invention can considerably increase the oral absorption of the active substances having an oral absorption rate as low as about 3% by about 5˜25 fold.

### EXPERIMENTAL EXAMPLES 8 TO 11

The dried sample prepared in Preparation Example 4 was taken in an amount corresponding to 40 mg/kg of the active substance, based on the body weight (kg) of test animals (Sprague-Dawley rats, 6˜8 week old male), and then was dissolved in water with stirring to prepare four drug solutions in such amounts that the total dose reached 0.5 ml. To each drug solution, saccharose distearate (HLB 7), sugar mono-distearate (HLB 11), sugar monostearate (HLB 15) and sugar monolaurate (HLB 16) were added in an amount of 50 mg, and dissolved. The abdomen of the test animals was surgically cut open and a polyethylene tube was inserted into duodenum through the lower portion of the stomach. After 0.5 ml of each drug solution was administered into the upper portion of the duodenum through the tube. The bioavailability was calculated in the same manner as in Experimental Examples 2 to 7.

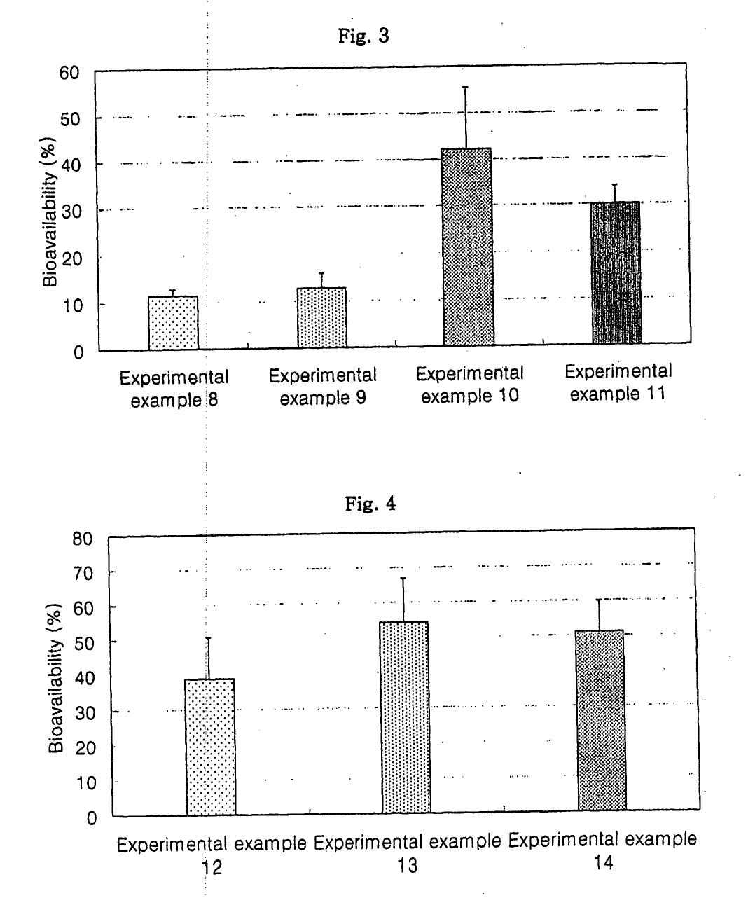

A graph showing the bioavailability (%) of the composition according to the present invention after administration to the duodenum of the test animals in accordance with Experimental Examples 8 to 11, is shown in FIG. 3. As is evident from the graph, the composition of the present invention exhibited a high bioavailability ranging from 10 to 40%. That is, the composition of the present invention can considerably increase the oral absorption of the active substance having an oral absorption rate as low as about 3% by about 3˜12 fold.

### EXPERIMENTAL EXAMPLES 12 TO 14

The dried samples prepared in Preparation Examples 1, 5 and 6 were taken in an amount corresponding to 40 mg/kg of the active substances, based on the body weight (kg) of test animals (Sprague-Dawley rats, 6˜8 week old male), and then were dissolved in water with stirring to prepare drug solutions in such amounts that the total dose reached 0.5 ml. 50 mg of sugar monolaurate (HLB 16) was added to the drug solutions of Experimental Examples 12 and 14, and 50 mg of sugar monostearate (HLB 15) was added to the drug solution of Experimental Example 13. The resulting mixtures were dissolved. The abdomen of the test animals was surgically cut open and a polyethylene tube was inserted into duodenum through the lower portion of the stomach. After 0.5 ml of each drug solution was administered to the upper portion of the duodenum through the tube, the abdomen was sutured. The bioavailability was calculated in the same manner as in Experimental Examples 2 to 7.

A graph showing the bioavailability (%) of the composition according to the present invention after administration to the duodenum of the test animals in accordance with Experimental Examples 12 to 14, is shown in FIG. 4. As shown in FIG. 4, the composition of the present invention exhibited a high bioavailability ranging from 35 to 55%. That is, the composition of the present invention can considerably increase the oral absorption of the active substances having an oral absorption rate as low as about 3% by about 11˜18 fold.

### EXPERIMENTAL EXAMPLES 15 TO 0.18

The dried samples prepared in Preparation Examples 7 to 10 were taken in an amount corresponding to 46 mg/kg of the active substance, based on the body weight (kg) of test animals (Sprague-Dawley rats, 6˜8 week old male), and then were dissolved in water with stirring to prepare drug solutions in such amounts that the total dose reached 0.5 ml. 25 mg of sugar monolaurate (HLB 16) was added to the drug solution of Experimental Example 15, and 12.5 mg of sugar monolaurate (HLB 16) was added to the drug solutions of Experimental Examples 16 to 18. The resulting mixtures were dissolved. The abdomen of the test animals was surgically cut open and a polyethylene tube was inserted into duodenum through the lower portion of the stomach. After 0.5 ml of each drug solution was administered to the upper portion of the duodenum through the tube, the abdomen was sutured. The bioavailability was calculated in the same manner as in Experimental Examples 2 to 7.

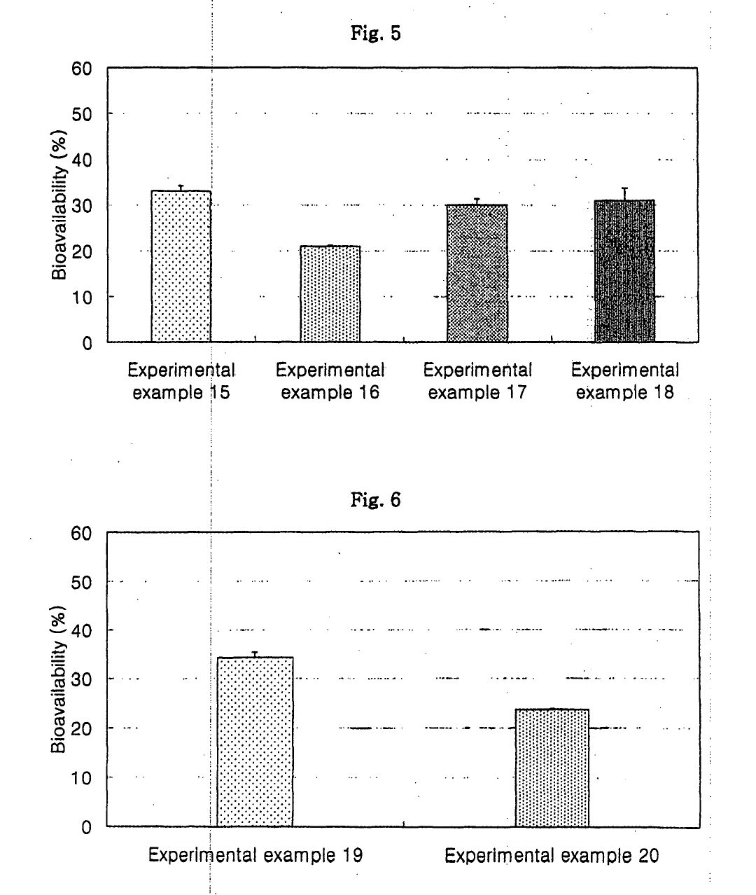

A graph showing the bioavailability (%) of the composition according to the present invention after administration to the duodenum of the test animals in accordance with Experimental Examples 15 to 18, is shown in FIG. 5. As shown in FIG. 5, the composition of the present invention exhibited a high bioavailability ranging from 20 to 35%. That is, the composition of the present invention can considerably increase the oral absorption of the active substance having an oral absorption rate as low as about 3% by about 7˜11 fold.

### EXPERIMENTAL EXAMPLES 19 AND 20

The dried samples prepared in Preparation Examples 11 and 12 were taken in an amount corresponding to 40 mg/kg of the active substance, based on the body weight (kg) of test animals (Sprague-Dawley rats, 6˜8 week old male), and then were dissolved in water with stirring to prepare drug solutions in such amounts that the total dose reached 0.5 ml. The abdomen of the test animals was surgically cut open and a polyethylene tube was inserted into duodenum through the lower portion of the stomach. After 0.5 ml of each drug solution was administered to the upper portion of the duodenum through the tube, the abdomen was sutured. The bioavailability was calculated in the same manner as in Experimental Examples 2 to 7.

A graph showing the bioavailability (%) of the composition according to the present invention after administration to the duodenum of the test animals in accordance with Experimental Examples 19 and 20, is shown in FIG. 6. As shown in FIG. 6, the composition of the present invention exhibited a high bioavailability ranging from 20 to 35%. This result demonstrates that since the organic alkalizing agent having a fatty acid structure has surface-activity due to its structural characteristics, the oral absorption rate can be increased without the addition of an additional surfactant. The composition of the present invention can considerably increase the oral absorption of the active substance having an oral absorption rate as low as about 3% by about 7˜11 fold.

### COMPARATIVE EXPERIMENTAL EXAMPLES 1 TO 4

In Comparative Experimental Examples 1 and 2, 0.5 ml of the drug solutions prepared in Comparative Preparation Examples 1 and 2, respectively, was used. In Comparative Experimental Example 3, the dried sample prepared in Preparation Example 1 was taken in an amount corresponding to 40 mg/kg of the active substance, based on the body weight (kg) of test animals (Sprague-Dawley rats, 6˜8 week old male), and then were dissolved in water with stirring to prepare a drug solution in such an amount that the total dose reached 0.5 ml. In Comparative Experimental Example 4, 0.5 ml of the drug solution prepared in Comparative Preparation Example 2 was used.

In Comparative Experimental Examples 1 to 3, the abdomen of the test animals was surgically cut open and a polyethylene tube was inserted into duodenum through the lower portion of the stomach. 0.5 ml of each drug solution was administered to the upper portion of the duodenum through the tube, except that 0.2 ml of glycerol caprylate was further administered to the upper portion of the duodenum in Comparative Experimental Example 4. The bioavailability was calculated in the same manner as in Experimental Examples 2 to 7.

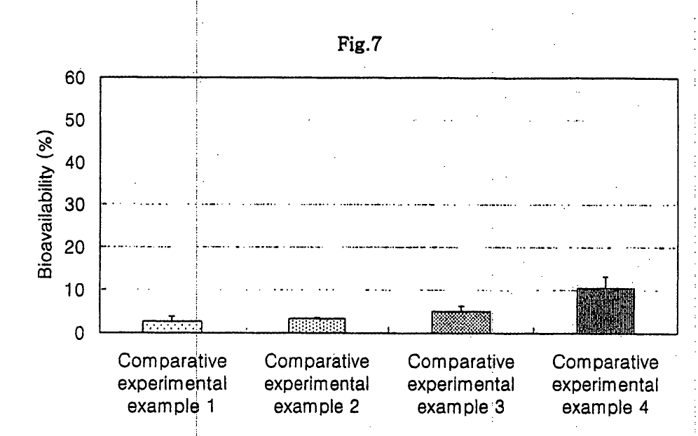

A graph showing the bioavailability (%) of the comparative compositions after administration to the duodenum of the test animals in accordance with Comparative Experimental Examples 1 to 4, is shown in FIG. 7. As shown in FIG. 7, in Comparative Experimental Examples 1 and 2 in which the active substances (drugs) were suspended or dissolved in accordance with general procedures, the bioavailability was as low as about 3%, indicating that the active substances cannot be orally absorbed. In addition, in Comparative Experimental Examples 3 and 4 in which the organic alkalizing agent was used alone and the surfactant was used alone, respectively, the bioavailability was as low as 5˜11%. This result reveals that only when the organic alkalizing agent and the surfactant having a fatty acid structure are simultaneously administered, or an alkalizing agent having the characteristics of both the organic alkalizing agent and the surfactant is administered, the oral absorption of the active substance can be considerably increased.

### INDUSTRIAL APPLICABILITY

As apparent from the above description, the pharmaceutical composition of the present invention comprises a polar active substance of which penetration through lipid membranes is nearly impossible, an organic alkalizing agent for neutralizing the charge of the polar active substance and reducing the polarity of the polar active substance, and a surfactant having a fatty acid structure. If necessary, instead of the organic alkalizing agent and the surfactant, another alkalizing agent having the characteristics of both the organic alkalizing agent and the surfactant may be used to increase the oral absorption rate. The present invention enables a great increase in the oral absorption rate of polar active substances which have been impossible to administer via the oral route, creating high value-added technologies. As the pharmaceutical composition according to the present invention can substitute oral dosage forms for injections of only injectable drugs, it removes inconvenience in using injections and ensures convenient use for patients.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A pharmaceutical composition for oral absorption of a polar active substance, consisting essentially of:
(a) at least one polar active substance having a bioavailability of less than 30% which is poorly absorptive through lipid membranes because of its high hydrophilicity and charged ion.
(b) at least one organic alkalizing agent having an amino acid or polyol structure which shows alkalinity in aqueous solution and is ionically bonded to the polar active substance; and
(c) at least one surfactant having a C₆₋₁₈ fatty acid structure which has an HLB (Hydrophilic-Lipophilic Balance) value of 4 to 18.

2. A pharmaceutical composition for oral absorption of a polar active substance, consisting essentially of:
(a) at least one polar active substance having a bioavailability of less than 30% which is poorly absorptive through lipid membranes because of its high hydrophilicity and charged ion.
(b) at least one organic alkalizing agent having a fatty acid ester structure which shows alkalinity in aqueous solution and is ionically bonded to the polar active substance.

3. The pharmaceutical composition according to claim 1, wherein the polar active substance is at least one selected from cephalordine, ceftiofur, cefixime, cefepime, cefoperazone, cefotaxime, ceftazidime, ceftriaxone, moxalactam, gentamicin, aztreonam, amikacin, isepamycin, netilmicin, tobramycin, vancomycin, daptomycin, teicoplanin, polymixin-B, bacitracin, heparin, parthyroid hormone, growth hormone and insulin.

4. The pharmaceutical composition according to claim 1, wherein the organic alkalizing agent having an amino acid structure is at least one selected from amino acids, amino acid derivatives and peptides; and the organic alkalizing agent having a polyol structure is at least one selected from alkaline saccharides, their oligomers and polymers prepared from 20 or fewer alkaline saccharides as monomers, and saccharide-like compounds.

5. The pharmaceutical composition according to claim 1, wherein the surfactant is at least one selected from sugar fatty acid esters, saccharin fatty acid esters, glycerol fatty acid esters, propylene glycol fatty acid esters, polyethylene glycol fatty acid esters, sorbitan fatty acid esters and polysorbitan fatty acid esters.

6. The pharmaceutical composition according to claim 2, wherein the organic alkalizing agent having a fatty acid ester structure is at least one alkaline substances prepared from the dehydration between a hydroxyl group (—OH) of a fatty acid ester and a carboxyl group (—COOH) of an amphoteric compound having both an amine group (—NH₂) and a carboxyl group (—COOH).

7. The pharmaceutical composition according to claim 1, wherein the active substance and the organic alkalizing agent are present in a charge ratio of 10:1˜1:10.

8. The pharmaceutical composition according to claim 1, wherein the polar active substance has at least one anionic moiety and has a partition coefficient (Log P) of 1.5 or lower.

9. The pharmaceutical composition according to claim 1, wherein the polar active substance and the organic alkalizing agent are combined with each other to form a hydrophobic conjugate having a size of 10 nm to 100 μm in water phase.

10. The pharmaceutical composition according to claim 1, further comprising at least one pharmaceutically acceptable excipient selected from disintegrating agents, suspending agents, thickening agents, lubricating agents, sweetening agents, plasticizers and preservatives.

11. The pharmaceutical composition according to claim 1, wherein the active substance forms a hydrophobic conjugate by intestinal juices after orally administered in the solid states.

12. The pharmaceutical composition according to claim 1, wherein the composition is formulated into syrups, dry syrups, powdery granules, tablets or capsules.

13. The pharmaceutical composition according to claim 11, wherein the composition is enteric coated when the active substance is unstable to gastric acid.
